(19) United States
(12) Patent Application Publication
(10) Pub. No.: US 2021/0322674 A1
(43) Pub. Date: Oct. 21, 2021

(54) DELIVERY OF MULTIPLE FLUIDS FROM MULTIPLE FLUID PUMPS

(71) Applicant: Ivenix, Inc, North Andover, MA (US)

(72) Inventors: George W. Gray, North Andover, MA (US); Marsden P. Marshall, Byfield, MA (US); Jason M. Baldwin, Portsmouth, NH (US); Christopher F. Pastore, Exeter, NH (US); Kimberly A. Iacoviello, Danvers, MA (US); Stephen C. Anthony, North Andover, MA (US)

(21) Appl. No.: 17/231,859

(22) Filed: Apr. 15, 2021

Related U.S. Application Data
(60) Provisional application No. 63/012,396, filed on Apr. 20, 2020.

(51) Int. Cl. A61M 5/168; A61M 5/142; G16H 20/17

(52) U.S. Cl. CPC A61M 5/16827; A61M 5/142; A61M 2005/14208; A61M 2205/583; G16H 20/17

(57) ABSTRACT

A fluid delivery system includes a fluid delivery controller and multiple fluid pumps. The multiple fluid pumps include a first fluid pump and a second fluid pump. A caregiver receives a fluid delivery order specifying a schedule of delivering multiple different fluids to a recipient. To deliver the different multiple different fluids to the recipient, the caregiver assigns each of the multiple fluid pumps to deliver a different fluid as specified by the fluid delivery order. One or more fluid delivery controllers then controls delivery of the multiple different fluids from the multiple fluid pumps in a manner as specified by the schedule.

### RELATED APPLICATION

This application claims the benefit of earlier filed U.S. Patent Application Ser. No. 63/012,396 entitled “SCHEDULED CONTROL OF DELIVERING MULTIPLE FLUIDS,” (Attorney Docket No. FLU20-02P), filed on Apr. 20, 2020, the entire teachings of which are incorporated herein by this reference.

### BACKGROUND

Conventional infusion pumps enable a caregiver to intravenously deliver fluid to a patient. For example, a physician first generates a medication order specifying one or more fluid-based drugs for delivery to a particular patient in a hospital. Typically, a pharmacy in the hospital receives the medication order supplied by the physician. In accordance with the medication order, the pharmacy dispenses a corresponding physical order by providing the drugs to a caregiver for delivery to a respective patient.

In certain instances, the medication order includes multiple fluid-based drugs that need to be administered intravenously to the respective patient. In such an instance, and assuming that each of the fluid-based drugs must be delivered by a separate fluid delivery system (fluid pump), the caregiver overseeing the patient must locate multiple fluid delivery systems (fluid pumps) in the hospital. This itself may be a difficult task because the hospital may not keep good track of unused medical equipment.

Subsequent to locating the multiple fluid delivery systems, the caregiver must transport the fluid delivery systems to a site where the patient resides. At the patient's bedside, in accordance with the medication order information, the caregiver manually operates the one or more fluid delivery systems to intravenously deliver the corresponding prescribed fluid-based drugs to the patient. Each fluid pump must be individually programmed by the caregiver to intravenously dispense a corresponding fluid to the patient.

Infusion devices are necessary components in the delivery of drip medications, fluid mixtures, and fluid hydration to patients. This is especially true for complex and dangerous therapies such as chemotherapies, which involve multiple drip medications and fluids which are delivered from multiple infusion devices and in a particular sequence over a fixed period of time.

Although the protocol for a fluid delivery therapy is often well defined from the start, delivery of said therapy sometimes involves multiple interactions with infusion devices so that the intended fluid therapy (infusate) is administered from different pumps at the appropriate time to the patient. Each of these manual interactions is an opportunity for error where a clinician could inadvertently change the sequence of the infusions, deliver the wrong drug at the wrong rate or forget to administer one of the fluids or medications in the protocol. In addition, each of these transitions is time consuming and could result in a medication error, a delay of therapy, or could extend the time required to administer the entire therapy.

### BRIEF DESCRIPTION OF EMBODIMENTS

Conventional techniques of intravenously delivering fluid to a patient suffer from deficiencies. For example, as previously discussed, operations of managing delivery of one or more fluids to a patient are tedious and can result in fluid delivery errors.

As a specific example, assume that a caregiver is required to administer infusion of two different types of prescribed fluids to a corresponding patient. First, as mentioned, the caregiver must retrieve two fluid pumps—one to deliver each of the prescribed fluid-based medicines. Thereafter, each of the fluid delivery systems has to be configured or programmed to deliver one of the multiple prescribed fluids.

In certain instances, to learn more about the different drugs that have been prescribed to the patient, and in view of specific details of how to administer the drugs, the caregiver has to log onto and provide input to a remote computer separate from the fluid pump. The remote computer is typically at least several feet away from the actual fluid pump that is being configured to deliver the prescribed fluids to the patient. In a case of administering multiple fluids to a patient, this means that, in order to fulfill delivery of a respective medication order, the caregiver has to program (such as provide input) to at least three different devices including a remote computer, a first fluid pump, and a second fluid pump. Thus, the caregiver must provide input to three different user interfaces, none of which may be aware of each other. The caregiver must be trained how to use each of the user interfaces—the user interface on the fluid pump and the user interface of the remote computer.

Failure to properly deliver the prescribed fluid-based drugs to a corresponding patient may be harmful, or possibly fatal. In contrast to conventional techniques, embodiments herein include unique ways of managing fluid delivery systems and related data, facilitating delivery of fluid to a recipient.

Embodiments herein include implementation of a unique fluid delivery schedule of delivering multiple fluids to a recipient at appropriate times via multiple fluid pumps. In one embodiment, the automated fluid delivery system enables clinicians (caregivers or other suitable entity) the ability to validate that the fluid medications are delivered to the right patient at the right time and in the correct sequence. Additionally, embodiments herein provide the ability to monitor the status of the therapy from a remote location and to pause its delivery at any point in the process.

As a more specific example, a fluid delivery system as discussed herein includes a fluid management system (such as one or more controllers) and multiple fluid pumps. The multiple fluid pumps include any number of fluid pumps such as a first fluid pump and a second fluid pump. A fluid delivery management system such as a controller receives a fluid delivery order specifying a schedule of delivering multiple different fluids to a recipient. In response to receiving the order, the fluid delivery management system (controller) assigns each of the multiple fluid pumps to deliver a different fluid as specified by the fluid delivery order. The fluid delivery management system (controller) then controls delivery of the multiple different fluids from the multiple fluid pumps in a manner as specified by the schedule.

The multiple different fluids include any number of fluids. In one embodiment, the multiple different fluids include a first fluid and a second fluid. The multiple fluid pumps include any number of fluid pumps. In one embodiment, the multiple fluid pumps assigned to execute the fluid delivery order include a first fluid pump and a second fluid pump.

In yet further example embodiments, the fluid delivery order indicates timing of delivering the second fluid from the second fluid pump with respect to delivering the first fluid from the first fluid pump.

In accordance with further example embodiments, the first fluid pump provides notification of a trigger event such as completion of delivering the first fluid to the second fluid pump. In such an instance, in accordance with the schedule, the notification of completion from the first fluid pump or other suitable entity triggers the second fluid pump to deliver the second fluid to the recipient.

Each of the multiple fluid pumps potentially includes a display screen. In one embodiment, the first fluid pump includes a display screen that displays a visual notification. The displayed visual notification indicates the schedule of delivering one or more of the multiple different fluids by each of the multiple fluid pumps.

Yet further embodiments herein include, displaying a notification on a display screen of a first fluid pump of the multiple fluid pumps. The notification indicates trigger conditions in which the second fluid pump is scheduled to deliver the second fluid with respect to the first fluid to the recipient.

In accordance with further example embodiments, controlling delivery of the multiple different fluids from the multiple fluid pumps in a manner as specified by the schedule includes: distributing the schedule to each of the multiple fluid pumps; and synchronizing operation of the multiple fluid pumps to a common clock. The fluid pumps collectively operate to provide the multiple fluids to the recipients.

Yet further embodiments herein include receiving the schedule from a communication device operated by a caregiver. The caregiver produces the schedule to indicate timing of each of the multiple fluid pumps delivering a respective fluid to the recipient.

In one nonlimiting example embodiment, as previously discussed, the multiple different fluids include a first fluid and a second fluid. The schedule indicates that delivery of the second fluid to the recipient is contingent upon delivery of the first fluid to the recipient. For example, in one embodiment, the schedule indicates to sequentially deliver the first fluid and the second fluid to the recipient such as one after the other. Alternatively, the fluid delivery schedule indicates to concurrently deliver the first fluid and the second fluid to the recipient, but that both the first fluid and the second fluid must be delivered simultaneously.

Yet further embodiments herein include, as specified by the schedule, delivering the second fluid to the recipient after a predetermined delay time following completion of delivering the first fluid to the recipient.

As previously discussed, the multiple fluid pumps can include a first fluid pump and a second fluid pump. Assignment of the multiple fluid pumps to execute the fluid delivery order includes: via input from a caregiver administering the fluid delivery order: i) selecting a first fluid pump to deliver a first fluid of the multiple different fluids, and ii) selecting a second fluid pump to deliver a second fluid of the multiple different fluids.

These and other more specific embodiments are disclosed in more detail below.

Note that any of the resources as discussed herein can include one or more computerized devices, fluid delivery systems, servers, base stations, wireless communication equipment, communication management systems, workstations, handheld or laptop computers, or the like to carry out and/or support any or all of the method operations disclosed herein. In other words, one or more computerized devices or processors can be programmed and/or configured to operate as explained herein to carry out different embodiments of the invention.

Yet other embodiments herein include software programs to perform the steps and operations summarized above and disclosed in detail below. One such embodiment comprises a computer program product including a non-transitory computer-readable storage medium (i.e., any physical computer readable hardware storage medium) on which software instructions are encoded for subsequent execution. The instructions, when executed in a computerized device (e.g., computer processing hardware) having a processor, program and/or cause the processor to perform the operations disclosed herein. Such arrangements are typically provided as software, code, instructions, and/or other data (e.g., data structures) arranged or encoded on a non-transitory computer readable storage medium such as an optical medium (e.g., CD-ROM), floppy disk, hard disk, memory stick, etc., or other a medium such as firmware, in one or more ROM, RAM, PROM, etc., or as an Application Specific Integrated Circuit (ASIC), etc. The software or firmware or other such configurations can be installed onto a computerized device to cause the computerized device to perform the techniques explained herein.

Accordingly, embodiments herein are directed to a method, system, computer program product, etc., that supports operations as discussed herein.

One embodiment herein includes a computer readable storage medium and/or system having instructions stored thereon. The instructions, when executed by computer processor hardware, cause the computer processor hardware to: receive a fluid delivery order specifying a schedule of delivering multiple different fluids to a recipient; assign a collection of multiple fluid pumps to execute the fluid delivery order of delivering the multiple different fluids to the recipient; and control delivery of the multiple different fluids from the multiple fluid pumps in a manner as specified by the schedule.

The ordering of the operations above has been added for clarity sake. Note that any of the processing steps as discussed herein can be performed in any suitable order.

Other embodiments of the present disclosure include software programs and/or respective hardware to perform any of the method embodiment steps and operations summarized above and disclosed in detail below.

It is to be understood that the system, method, apparatus, instructions on computer readable storage media, etc., as discussed herein also can be embodied strictly as a software program, firmware, as a hybrid of software, hardware and/or firmware, or as hardware alone such as within a processor, or within an operating system or within a software application.

As discussed herein, techniques herein are well suited for managing and facilitating use of medical devices. However, it should be noted that embodiments herein are not limited to use in such applications and that the techniques discussed herein are well suited for other applications as well.

Additionally, note that although each of the different features, techniques, configurations, etc., herein may be discussed in different places of this disclosure, it is intended, where suitable, that each of the concepts can optionally be executed independently of each other or in combination with each other. Accordingly, the one or more present inventions as described herein can be embodied and viewed in many different ways.

Also, note that this preliminary discussion of embodiments herein purposefully does not specify every embodiment and/or incrementally novel aspect of the present disclosure or claimed invention(s). Instead, this brief description only presents general embodiments and corresponding points of novelty over conventional techniques. For additional details and/or possible perspectives (permutations) of the invention(s), the reader is directed to the Detailed Description section and corresponding figures of the present disclosure as further discussed below.

### BRIEF DESCRIPTION OF THE DRAWINGS

FIG. 1 is an example diagram illustrating management of delivering a fluid therapy of multiple different fluids from multiple fluid pumps to a recipient according to embodiments herein.

FIG. 2 is an example diagram illustrating a fluid order and generation of fluid delivery schedule information according to embodiments herein.

FIG. 3 is an example diagram illustrating display of delivery status information associated with delivery of fluid therapy from multiple fluid pumps according to embodiments herein.

FIG. 4 is an example diagram illustrating communications amongst multiple fluid pumps during a fluid delivery according to embodiments herein.

FIG. 5 is an example diagram illustrating display of delivery status information associated with delivery of fluid therapy from multiple fluid pumps according to embodiments herein.

FIG. 6 is an example diagram illustrating communication of messages associated with a fluid delivery according to embodiments herein.

FIG. 7 is an example diagram illustrating display of delivery status information associated with delivery of fluid therapy from multiple fluid pumps according to embodiments herein.

FIG. 8 is an example diagram illustrating a computer architecture in which to execute one or more embodiments as discussed herein.

FIG. 9 is an example diagram illustrating a method according to embodiments herein.

The foregoing and other objects, features, and advantages of the invention will be apparent from the following more particular description of preferred embodiments herein, as illustrated in the accompanying drawings in which like reference characters refer to the same parts throughout the different views. The drawings are not necessarily to scale, with emphasis instead being placed upon illustrating the embodiments, principles, concepts, etc.

### DETAILED DESCRIPTION AND FURTHER SUMMARY OF EMBODIMENTS

A fluid delivery system includes a fluid management system (such as a single controller or multiple controllers) and multiple fluid pumps. The multiple fluid pumps include any number of fluid pumps such as a first fluid pump and a second fluid pump. The fluid management system receives a fluid delivery order specifying multiple different fluids to deliver to a recipient. In response to receiving the order, in accordance with generated schedule information, the fluid management system assigns each of the multiple fluid pumps to deliver a different fluid as specified by the fluid delivery order. The schedule information distributed to the multiple fluid pumps controls delivery of the multiple different fluids from the multiple fluid pumps.

Accordingly, embodiments herein include methods, systems, etc., for delivering a therapy involving multiple infusions in an orchestrated and synchronized fashion across multiple independent infusion devices and through one or more fluid paths available on those devices.

Note further that one embodiment herein provides a means to define the individual infusions associated with an infusion therapy, define when each of those infusions should start, and optionally end or change, in support of that therapy, define the devices, and optionally the fluid paths, through which the infusion will be delivered and the events that initiate the start of each infusion.

Once started, the fluid therapy can be delivered in part or in its entirety across multiple infusion devices and without the need for human interaction, reducing the chance of human error and streamlining the administration of the therapy. In order to accomplish this, in one embodiment, the infusion devices align around a common therapy to independently deliver specific infusions within the therapy in a synchronized manner while concurrently verifying proper execution of the therapy delivery being performed by peer infusion devices.

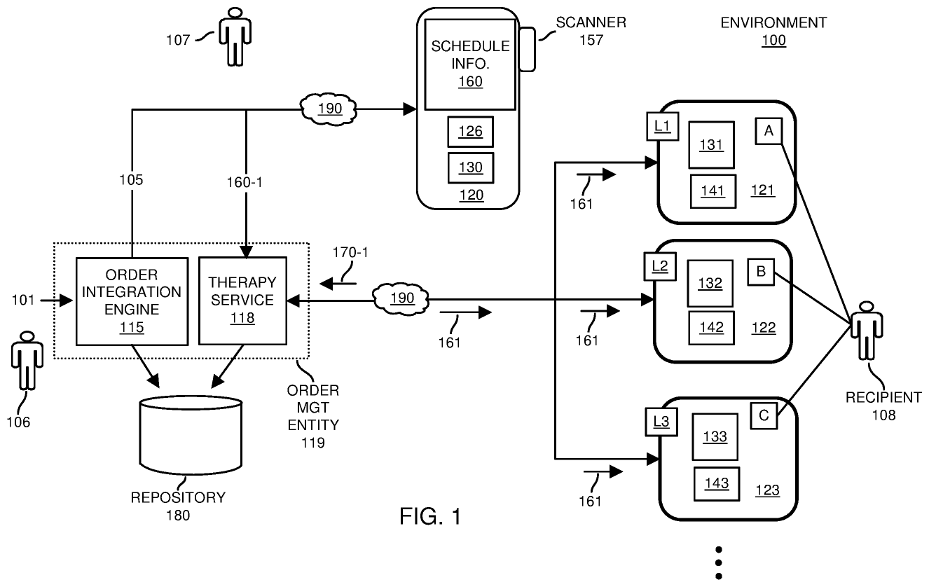

Now, more specifically, FIG. 1 is an example diagram illustrating management of delivering multiple fluid pumps to a recipient via multiple different fluid pumps according to embodiments herein.

Embodiments herein include systems and methods for delivering an infusion therapy protocol in a coordinated manner via multiple, independent infusion devices (fluid pumps 121, 122, 123, etc.) operated in accordance with a delivery schedule.

As a more specific example, a fluid delivery system 100 includes multiple fluid pumps. The multiple fluid pumps include any number of fluid pumps such as fluid pump 121, fluid pump 122, fluid pump 123, etc.

The fluid management system 126 (a.k.a., therapy definition manager) of communication device 120 receives fluid order 105 such as specifying multiple fluids A, B, C, etc., and a schedule information indicating a timing and corresponding rates of delivering the multiple different fluids A, B, C, etc., to a recipient 108.

In one embodiment, the caregiver 106 such as a doctor, nurse, etc., prescribes a respective fluid therapy of one or more fluids. The fluid management system 126 receives the fluid order 105 from the order integration engine 115 or other suitable entity.

For example, in response to receiving the fluid order 105 (including fluid order information), the caregiver 107 operates the fluid delivery management system 126 (such as controller or other suitable entity) associated with communication device 120 to assign each of the multiple fluid pumps a task of delivering a different fluid as specified by the fluid delivery order.

In one embodiment, the communication device 120 is disparately located with respect to the fluid pumps and the order management entity 119. However, note that the communication device 126 can be integrated into one or more of the fluid pumps 121, 122, 123, etc.

In further example embodiments, the caregiver 107 operates the communication device 120 to scan one or more labels (such as label L1 affixed to the fluid pump 121, label L2 affixed to the fluid pump 120, label L3 affixed to the fluid pump 123) associated with each of the fluids during the assignment. In this manner, the communication device 120 and corresponding fluid management system 126 receive notification of a group of multiple available fluid pumps that have been acquired to deliver the fluid therapy as specified by the fluid order 105.

In one embodiment, the received fluid order 105 specifies the need to deliver multiple fluids including fluid type A, fluid type B, fluid type C, etc. The caregiver 107 or other suitable entity assigns each of the fluid pumps in the group (as created by the caregiver 107 or other suitable entity) to deliver a respective selected fluid as specified by the fluid order 115.

Via the fluid management system 126 executed on the communication device 120, assume that the caregiver 107 assigns (selects) the fluid pump 121 to deliver fluid A to recipient 108; the caregiver 107 assigns (selects) the fluid pump 122 to deliver fluid B to recipient 108; the caregiver 107 assigns Selects) the fluid pump 123 to deliver fluid C to recipient 108; and so on. In one embodiment, the caregiver creates the association between the respective fluid pump and the fluid based on input from the caregiver 107.

Additionally, the caregiver 107 or fluid management system 126 creates a schedule 160-1 (such as fluid delivery control information derived from order 105 or newly generated by the caregiver 107 or a combination of both) indicating timing of delivering each of the fluids A, B, C, etc., associated with the order 105.

For example, assume that the fluid management system 126 produces the schedule information 160-1 to indicate delivery of fluid A between time T1 and time T2; the fluid management system 126 produces the schedule information 160-1 to indicate delivery of fluid B between time T3 and time T4; the fluid management system 126 produces the schedule information 160-1 to indicate delivery of fluid C between time T5 and time T6; and so on.

Note that, according to the schedule information 160-1, the fluids can be delivered simultaneously, partially overlapping, one after the other, etc.

As previously discussed, in one embodiment, the fluid order 105 indicates timing of delivering the second fluid B from the second fluid pump 122 with respect to delivering the first fluid A from the first fluid pump 121; the fluid delivery order 105 indicates timing of delivering the third fluid C from the third fluid pump 123 with respect to delivering the second fluid B from the second fluid pump 122; and so on.

The fluid management system 126 of communication device 120 communicates the schedule information 160-1 (a.k.a., delivery control information) that subsequently controls delivery of the multiple different fluids A, B, C, etc., from the multiple fluid pumps 121, 122, 123, etc., in accordance with an appropriate schedule.

In accordance with further example embodiments, the schedule information 160-1 indicates contingencies of delivering the fluids relative to each other. For example, in one embodiment, the schedule information 160-1 indicates that delivery of the second fluid B to the recipient 108 is contingent upon a condition of completing delivery of the first fluid A to the recipient; the schedule information 160-1 indicates that delivery of the third fluid C to the recipient 108 is contingent upon a condition of delivering the first fluid A or the second fluid B to the recipient 108.

In accordance with another example embodiment, the schedule information 160-1 can be configured to indicate to concurrently deliver the first fluid A and the second fluid B to the recipient 108.

Alternatively, note that the schedule information 160-1 can be configured to indicate to sequentially deliver the first fluid A and the second fluid B to the recipient 108 such as one after the other in a manner as previously discussed.

Yet further embodiments herein include, as specified by the schedule information 160-1, delivering the second fluid B to the recipient after a predetermined delay time following completion of delivering the first fluid A to the recipient 108.

In accordance with further example embodiments, controlling delivery of the multiple different fluids A, B, C, etc., from the multiple fluid pumps in a manner as specified by the schedule 160-1 includes: distributing the schedule information 160-1 to each of the multiple fluid pumps 121, 122, 123, etc., and synchronizing operation of each of the multiple fluid pumps to operate off a common master clock.

In one embodiment, the communication device 120, order management entity 119, or other suitable entity communicates the schedule information 160-1 over network 190 via communications 161 to each of the more of the fluid pumps 121, 122, 123, etc. Additionally, or alternatively, the schedule information 160-1 is communicated via communications 161 to a first fluid pump; the first fluid pump communicates the schedule information 160-1 to the other fluid pumps in the group.

In accordance with further example embodiments, fluid pump 121 includes controller 141 that implements delivery of the fluid A in accordance with the distributed schedule information 160-1; fluid pump 122 includes controller 142 that implements delivery of the fluid B in accordance with the distributed schedule information 160; fluid pump 123 includes controller 143 that implements delivery of the fluid C in accordance with the distributed schedule information 160-1; and so on.

In one embodiment, each of the different controllers in the assigned group of fluid pumps receives the same schedule information 160-1, which provides details or rules of delivering each of the fluids.

In accordance with further example embodiments, note that each of the controllers of the fluid pumps 121, 122, 123, etc., is operative to communicate with each other and potentially the order management entity 119 and communication device 120 regarding a status of delivering respective fluid according to the fluid order 105. For example, controller 141 communicates with controller 142 and controller 143 over a respective network or wireless communication link; controller 142 communicates with controller 141 and controller 143 over a respective network; controller 143 communicates with controller 141 and controller 142 over a respective network; and so on.

As previously discussed, delivery of fluid B to the recipient may be contingent upon delivery of fluid A. In one embodiment, the first fluid pump 121 and controller 141 provide notification of completion of delivering the first fluid A to the controller 142 of the second fluid pump 122. In such an instance, in accordance with the schedule information 160, the notification between controllers triggers the second fluid pump 122 to commence delivering the second fluid B to the recipient 108.

As further shown, each of the multiple fluid pumps 121, 122, 123, etc., includes a display screen. For example, fluid pump 121 includes display screen 131; fluid pump 122 includes display screen 132; fluid pump 123 includes display screen 133; and so on. Each of the display screens displays any suitable information associated with the delivery of fluid.

In one embodiment, the first fluid pump 121 includes a display screen 131 that displays one or more visual notifications associated with delivering fluid as specified by the fluid order 115. In one embodiment, the displayed visual notification indicates the schedule information 160-1 of delivering the multiple different fluids by each of the multiple fluid pumps. In a similar manner, each of the display screens 132, 133, etc., can be configured to display the respective schedule information 160-1.

Yet further embodiments herein include, via one or more of the display screens 131, 132, 133, etc., displaying a notification on one or more respective display screen indicating triggering conditions in which the respective fluid pump is scheduled to deliver (via an infusion) a corresponding fluid to the recipient with respect to another fluid supplied by another fluid pump.

In yet further example embodiments, the group of fluid pumps assigned to execute the fluid order 105 operate in an autonomous manner. For example, as previously discussed, each of the fluid pumps 121, 122, 123, etc., in the group receives the schedule information 160-1 (fluid delivery control information) over a communication link. The connectivity such as network 190 between the group of fluid pumps and the order management entity 119, communication device 120, etc., may be prone to failure. In one embodiment, the fluid pumps in the group communicate amongst each other to execute the respective fluid order 115 even though the group of fluid pumps are unable to communicate over network 190 with the order management entity 119 (such as a management server), communication device 120, etc. In other words, each of the fluid pumps communicates with each other over one or more communication links independent of communicating over network 190. In such an instance, after the schedule information 160-1 is distributed to the fluid pumps, the fluid pumps communicate with each other to fulfill the fluid delivery order to the recipient as indicated by the schedule information 160-1.

### Additional Details of Providing Fluid Therapy

As previously discussed, the environment 100 includes multiple components. For example, environment 100 includes an Order Integration Engine 115 (such as hardware, software, or a combination of hardware and software), receives and stores physician orders 101 related to fluid therapies being administered to the recipient 108 such as a patient. The environment 100 further includes a Therapy Definition Manager (such as fluid management system 126, executed as hardware, software, or a combination of hardware and software), which is operative to configure how a fluid therapy is to be delivered via multiple fluid pumps. In one embodiment, the environment 100 further includes a database such as stored in repository 180, which stores persistent information about the fluid therapy, including, but not limited to, the underlying orders, the means in which it is delivered as well as the current status of the therapy and each associated infusion.

Environment 100 further includes Therapy Service 118 (executed as hardware, software, or a combination of hardware and software). The therapy service 118 is operative to facilitate movement of information between the Therapy Definition

Manager, the Therapy Repository, and the infusion devices (a.k.a., fluid pumps 121, 122, 123, etc.).

Note that the environment 100 and corresponding order management entity 119 further includes Therapy Dashboard 170 operative to remotely monitor the fluid therapy after it begins.

As previously discussed, the environment 100 further includes intelligent infusion devices (such as fluid pump 121, 122, 123, etc.), each including one or more fluid delivery inlets to receive fluid for delivery. In one embodiment, the fluid pumps (a.k.a., infusion devices) are operative to retrieve specific details about delivering a fluid therapy from the Therapy Service 118. As further discussed herein, via the retrieved information such as schedule information 160-1 from the Therapy Service 118, the fluid pumps determine their individual role in the delivery of a multi-infusion therapy. The group of fluid pumps collectively work to deliver the appropriate infusions at the appropriate times and in the appropriate sequence to the recipient 108 (patient).

Further, as previously discussed, embodiments herein include using physician orders 101 acquired from a network connected order entry system or other network connected repository for these orders. The Order Integration Engine 115 (a.k.a., order integration engine) may acquire a single order for the entire fluid therapy, inclusive of the individual infusions that exist as part of that fluid therapy or it may receive a collection of individual orders which may be then be associated with a therapy through the use of the Therapy Definition Manager (such as fluid management system 126). Upon receipt of the orders, the Order Integration Engine 115 stores those orders in the Therapy Repository 180.

In further example embodiments, as previously discussed, the Therapy Definition Manager (such as fluid management system 126) implements functionality to identify the recipient patient, the infusion devices (fluid pumps) and the infusates (i.e. drip medications and fluid mixtures) associated with a fluid therapy as well as the predetermined sequence in which the infusates are to be delivered in accordance with schedule information. Note that any suitable method can be used to identify each fluid pump including the manual entry of identifiers or through the scanning of barcodes or I.D. tags associated with each.

Once the patient (such as recipient 108) is identified via the order, the Therapy Definition Manager (such as fluid management system 126) retrieves all unfulfilled orders associated with that patient (a.k.a. recipient 108) from the Therapy Repository 180 and presents those orders to the caregiver 107 (user) on a display screen 130 of the communication device 120.

Note that orders may exist for a specific therapy and contain sub-orders comprising one or more infusion orders. Independent orders may also exist for the infusions themselves.

After retrieval of the order 105 and corresponding information, the Therapy Definition Manager (fluid management system 126) implements functionality to select the orders that are associated with the fluid therapy (as specified by the fluid order 105) being administered to the recipient 108 and then organizes those orders into a sequence of infusions that reflect how the therapy should be delivered. In one embodiment, if desired, the Therapy Definition Manager (fluid management system 126) implements functionality to hide any orders that are not related to the therapy.

For each infusion (such as fluid type A, B, C, etc.) associated with the fluid order 105, the Therapy Definition Manager (fluid management system 126) defines program settings for the corresponding fluid pumps 121, 122, 123, etc., such as infusion devices and events that will cause the infusion to start on that fluid pump. The Therapy Definition manager (fluid management system 126) implements functionality to scan barcodes and/or other identifying tags for the infusate and use this information to verify that the scanned information matches the selected medication order.

Using the details of the order 105 for each infusion (A, B, C, etc.), the Therapy Definition Manager (fluid management system 126) derives a set of program settings and presents them to the caregiver 107. The caregiver 107 will have the option to modify these setting or accept them as is. These settings may include, but are not be limited to, the admixture or concentration of the medication in the fluid source, the dose, dose rate, rate, volume to be infused and duration of the infusion.

In one embodiment, each infusion in the therapy must have an initiating event which causes it to start. This could include clicking start on the infusion device, a delay timer expiring, the device's clock reaching a specific time, the start or end of another infusion defined as part of the therapy, the occurrence of an alarm that occurs during the administration of another infusion defined as part of the therapy or some other event message broadcast from the Therapy Service 118 or another infusion device (fluid pump) scheduled in the delivery of the therapy. The Therapy Definition Manager (fluid management system 126) provides the means to define this initiating event and, optionally, built in delays between the initiating event and the start of the infusion on a selected fluid pump.

In order to transfer the program settings to a respective infusion device, the infusion device is first identified. The Therapy Definition Manager (fluid management system 126) implements functionality to identify the fluid pump (infusion device) in a variety of ways, including, but limited to, entering the serial number of the device, scanning a barcode on the device or scanning some other I.D. tag located on or within the device. As previously discussed, in one embodiment, the communication device includes scanner 157 that scans respective labels L1, L2, L3, etc.

As previously discussed, the caregiver 107 or other suitable entity assigns a respective fluid in the order 105 to a fluid pump. Assignment indicates that the fluid pump is assigned to deliver the identified fluid.

After the fluid pumps are identified, and the schedule information 160-1 is generated, the Therapy Definition Manager communicates program settings (such as schedule information 160-1) to each of the infusion devices (fluid pumps). The program settings indicate assignment of a fluid to a respective fluid pump. Thus, the receiving fluid pump is able to identify a particular fluid it is going to deliver to the recipient as well as when or what conditions to deliver it.

More specifically, each respective receiving fluid pump uses the program settings, infusate information, patient identifier and initiating events (such as conditions of starting delivery of a respective fluid) to then program itself. After being programmed, the infusion device (fluid pump) is setup to deliver the infusate (fluid type) in a particular order relative to other infusates being administered as part of the fluid therapy.

The process of defining the specifics of each infusion in the therapy may continue until the entire fluid therapy is defined. At any time, an infusion device can begin administering an infusate. Likewise, additional medications can be added and all already defined infusions can be modified or removed from the therapy.

### Verifying the Therapy Setup

Note that, in one embodiment, a copy of the fluid pump identities and corresponding fluid pump settings are maintained in the Therapy Repository 180. As each infusion starts, the status of that infusion is also maintained in the repository via communications from the respective fluid pump delivering the fluid over network 190 to the order management entity 119. Via access to the information stored in repository 180, the caregiver 107, at their option, can review the status and details of the therapy definition process through the Therapy Definition Manager (manager 126).

In one nonlimiting example embodiment, the Therapy Definition Manager (126) includes functionality to command the already programmed infusion devices (fluid pumps) to display their own specific details such as the sequence in which the infusions shall occur, the events that will initiate the start of the infusion and the infusion program settings themselves. In one embodiment, each fluid pump includes a display screen to indicate details of the corresponding for review by the caregiver 107.

It should be noted that multiple infusions may be delivered in part or in their entirety from the same infusion device and fluid path. For example, an infusion device may have a large source container of fluid that is delivered in part between the administrations of medications from multiple fluid pumps. More specifically, in one nonlimiting example embodiment, 33% of fluid of a particular type can be delivered after the administration of each of three medications.

### Synchronized Infusion Delivery

Note further that the fluid pumps can be configured to discover each other on the local wired or wireless network and establish peer-to-peer group messaging directly with each other. For example, each of the fluid pumps can be configured to send out a discovery request to learn of other fluid pumps in the vicinity. The other pumps respond to the discovery request indicating their presence and availability in environment. Additionally, or alternatively, each of the fluid pumps can be configured to send a beacon indicating its presence to others. In accordance with the schedule information 160-1, each of the fluid pumps as indicated by the schedule information 160-1 communicates with other fluid pumps in the group to coordinate delivery of the fluid therapy as specified by the fluid order 105 and the schedule information 160-1.

Via the received schedule information 160-1, each fluid pump thus knows the identity of other fluid pumps assigned to the group that is going to deliver the fluid order 105 to the recipient 108. Based on being part of the same sequenced therapy and group as specified by the schedule information 160-1, the fluid pumps create wired or wireless connectivity with each other. The fluid pumps then use the connectivity to provide notifications to each other such as heartbeat signals indicating they are connected and alive, trigger events such as that the respective fluid pump has completed a delivery of its assigned fluid, synchronization information to maintain time synchronicity of the fluid pumps, and so on.

In one embodiment, the fluid pumps in the group can both broadcast messages to other fluid pump members of the group and send messages directly to another specific infusion device. If desired, communications can be secured (such as encrypted) to prevent malicious tampering.

Thus, in one embodiment, each fluid pump in the group of fluid pumps as specified by the schedule information 160-1 has the ability to communicate with other fluid pumps associated with the fluid therapy delivery without the need for an intermediary agent such as order management entity 119, communication device 120, etc. As previously discussed, in one embodiment, the group of fluid pumps has the ability to communicate with each other and collectively provide the fluid therapy as specified by the fluid order 105 without connectivity to the network 190. In other words, the group of assigned fluid pumps 121, 122, 123, etc., are part of an independent network supplying the respective fluid therapy to the recipient.

In one nonlimiting example embodiment, each of the fluid pumps 121, 122, 123, etc., is disparately located with respect to each other.

In further example embodiments, when network 190 is available, each of the fluid pumps in the group further has the ability to communicate with the Therapy Service 118 of the order management entity 119. As part of the setup of each fluid pump, the respective fluid pump ensures that it can communicate at least with any infusion devices it is dependent on or that depend on it (i.e., per its own initiating event definition). This may include a second device running an infusion that must complete prior to it starting or a third device running an infusion with which the first device is configured to run concurrently. As mentioned, communication amongst the fluid pumps may be established over any network, either wired or wireless.

After communication is established amongst the fluid pumps in the group, the dependent infusion device (fluid pump awaiting delivery of a fluid by another fluid pump) will request to be notified of all events. The upstream device (such as fluid pump 121 assigned to deliver a first fluid to the recipient before other fluids by fluid pumps 122, 1233, etc.) communicates its own status as well any notable events that have already occurred. For example, if the upstream fluid pump has already started infusing a respective fluid, it communicates such information to other fluid pumps in the group. Thus, each of the fluid pumps is aware of the status of all other fluid pumps collectively delivering fluid therapy to the recipient 108.

As the fluid therapy continues, the upstream fluid pumps (such as fluid pump that delivers first) and concurrent infusion devices (fluid pumps that simultaneously deliver fluid to the recipient 108 in accordance with the schedule information) continue to communicate their respective status and all significant events (i.e. start, stop, pause, change in settings, failures, alarm conditions, etc., associated with the fluid pump) to the downstream fluid pumps (e.g., fluid pumps 122, 123, etc., scheduled to deliver a respective fluid after the upstream fluid pump) and other concurrent infusion devices. Using this information, fluid pumps coordinate the start of their own infusion.

And, in the event that an upstream or concurrent fluid pump fails, communication is disrupted, or an upstream or concurrent infusion is cancelled, the downstream infusion device (such as fluid pump 122, 123) may terminate, pause indefinitely or even alarm (such as on display screen or audible alarm) to notify the caregiver 107 of the event. For example, an infusion device may terminate and generate an alarm if a concurrent infusion of respective fluid fails. A fluid pump also may pause indefinitely if an upstream fluid pump (such as fluid pump 121) is paused or the device fails.

In further example embodiments, if connectivity through network 190 to the order management entity 119 is available, each respective fluid pump communicates their status and significant trigger events to the Therapy Service 118, which will then store that information in the Therapy Repository 180.

The Therapy Service 118 may also be queried by any fluid pump regarding the status of any other fluid pump participating in the delivery of the fluid therapy as specified by the fluid order 105. This essentially acts as an alternate channel of communication or a secondary channel of communication to allow each infusion device to validate the information they are receiving from a second source, providing a high degree of security. In addition, the repository 180 is a source for additional information, providing each fluid pump information regarding the current state of delivering the fluid therapy.

### Monitoring of Ongoing Infusions

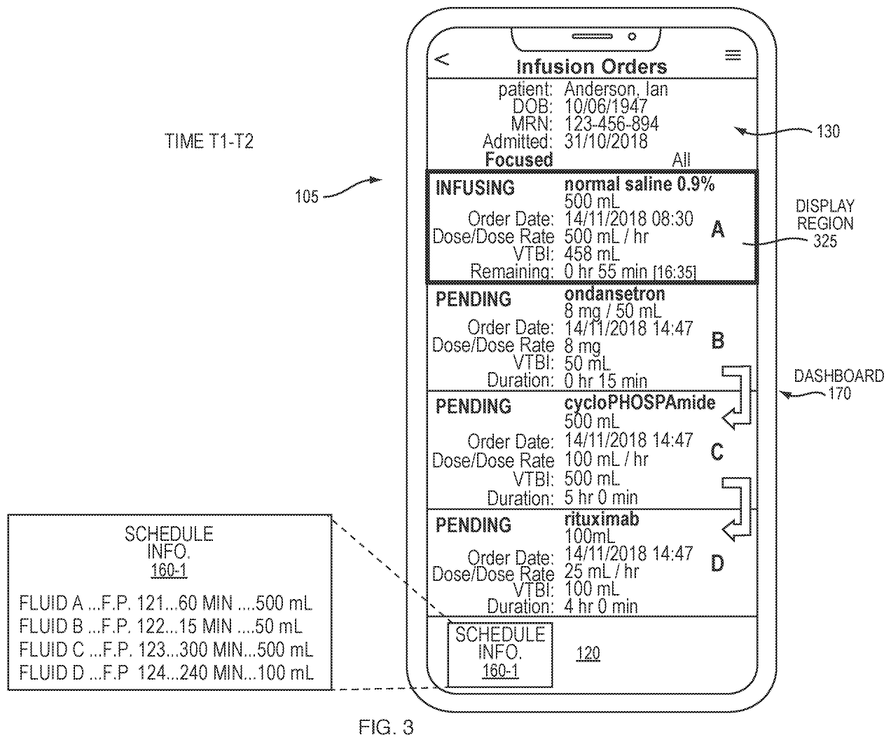

In further example embodiments, the status of a fluid therapy can be monitored by a clinician through the use of the Therapy Dashboard 170 in FIG. 3.

In one embodiment, the Dashboard 170 shows each infusion, the programmed infusion settings, an indication of the state of the infusion (e.g. completed, running, paused, pending) and any identified dependencies each infusion has with other infusions defined as part of the therapy

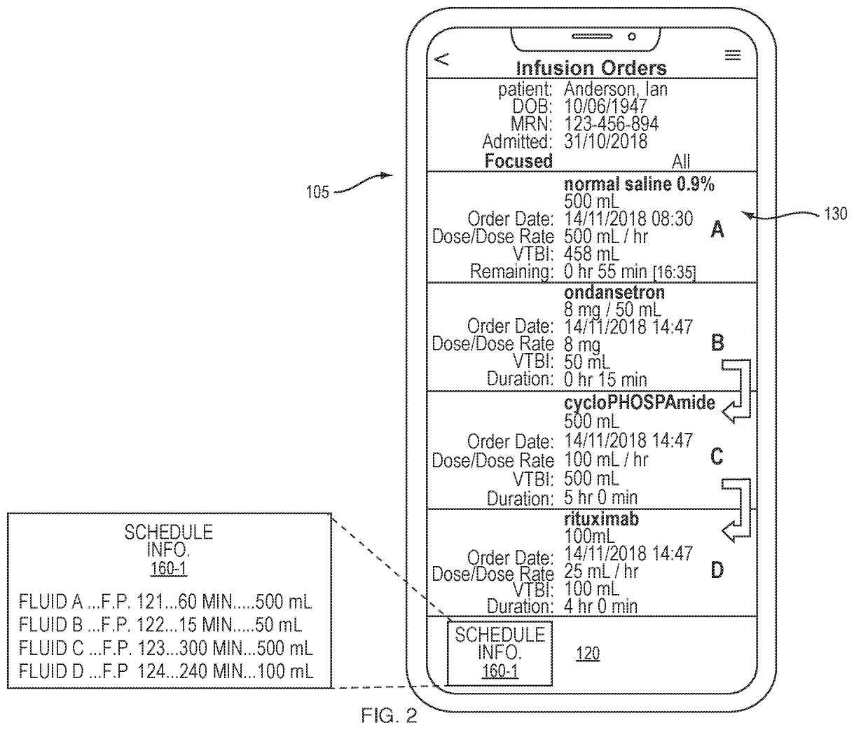

FIG. 2 is an example diagram illustrating a fluid order and generation of fluid delivery schedule information according to embodiments herein.

In this example embodiment, the caregiver 107 creates schedule information 160-1 (i.e., fluid delivery control information) of delivering multiple fluids to the respective recipient 108. Initially, the communication device 120 receives information associated with each fluid to be delivered in the fluid order 105 such as type of fluid, amount of fluid to be delivered, rate of delivering the fluid to recipient 108, dose/dose rate, order date, time duration of delivery, etc.

In one embodiment, the order information 105 includes data indicating the specific schedule (such as including sequential order in which to deliver the fluids as specified by the fluid therapy) that is used to deliver the different fluids.

In further example embodiments, to produce the schedule information 160-1, the caregiver 107 selects, and via fluid management system 126 (such as hardware, executed software, or a combination of hardware and executed software), the different fluid pumps 121, 122, 123, etc., that are to deliver each respective fluid A, B, C, etc., as specified by the order 105.

More specifically, as shown in generated schedule information 160-1, the caregiver 107 or other suitable entity assigns fluid A of the received fluid order 105 for delivery by fluid pump 121; the caregiver 107 or other suitable entity assigns fluid B of the received fluid order 105 for delivery by fluid pump 122; the caregiver 107 or other suitable entity assigns fluid C of the received fluid order 105 for delivery by fluid pump 123; the caregiver 107 or other suitable entity assigns fluid D of the received fluid order 105 for delivery by fluid pump 124; and so on.

In one embodiment, the fluid management system 126 of the communication device 120 initiates display of the identity of each fluid pump and corresponding fluid associated with fluid order 105 on the display screen 130. The caregiver 107 indicates which respective fluid pump is assigned to deliver each different fluid as above.

Referring again to FIG. 1, via communications 161, the communication device 120 communicates the generated schedule information 160-1 to each of the fluid pumps 121, 122, 123, etc.

As previously discussed, the fluid pumps 121 collectively operate to provide delivery of the different fluids A, B, C, D, etc., in accordance with the schedule information 160-1.

In response to receiving the schedule information 160-1 via communications 161, each of the fluid pumps initiates display of information regarding which of the multiple fluids associated with the fluid order 105 it has been assigned to deliver to the recipient 108.

For example, fluid pump 121 displays information on display screen 131 of the fluid pump 121 such as an identity of fluid A to be delivered to the recipient 108, rate of delivering the fluid A to recipient 108, dose/dose rate of fluid A, order date, time duration of delivery associated with respective fluid, etc.

Fluid pump 122 displays information on display screen 132 of the fluid pump 122 such as an identity of fluid B and corresponding information such as amount of fluid B to be delivered to the recipient 108, rate of delivering the fluid B to recipient 108, dose/dose rate of fluid B, order date, time duration of delivery associated with fluid B, etc.

Fluid pump 123 displays information on display screen 133 of the fluid pump 123 such as an identity of fluid C to be delivered to the recipient 108, rate of delivering the fluid C to recipient 108, dose/dose rate of fluid C, order date, time duration of delivery associated with fluid C, etc.

Thus, the caregiver 107 is informed of which fluid source (such as type A, B, C, etc.) to connect to each fluid pump.

FIG. 3 is an example diagram illustrating display of status information associated with multiple fluid pumps according to embodiments herein.

At time T1, assume that the caregiver 107 or other suitable entity initiates execution of fluid order 105 as specified by the schedule information 160-1. In response to receiving input (such as from caregiver 107 through the fluid pump itself or the communication device 120) to begin the infusion, the fluid pump 121 starts delivery of the fluid A to the recipient 108.

In this example embodiment, each of the fluid pumps 121, 122, etc., displays a current status of the pump delivering a respective fluid to the recipient 108. For example, display screen 131 of the fluid pump 121 displays a status of delivering fluid A to the recipient 108; display screen 132 of the fluid pump 122 displays a status of delivering fluid B to the recipient 108; and so on.

In further example embodiments, each of the fluid pumps (such as via respective controllers) communicate with each other to indicate their status as well as communicate with the order management entity 119 and/or communication device 120 indicating the status of delivering the fluids. In one embodiment, between time T1 and T2 (time period such as 1 hour in which to deliver fluid A), the fluid pump 121 communicates a status of the delivery to communication device 120. In response to receiving the input, the communication device 120 displays a notification in display region 325 of the display screen 130 indicating the status of delivering the fluid A. In one embodiment, the fluid pump 121 also displays information in display region 325 on the display screen 131 of the fluid pump 121.

Thus, the caregiver 107 can view the display screen 130 of the communication device 120 or the display screen 131 of the fluid pump 121 to determine a status of delivering the respective fluids.

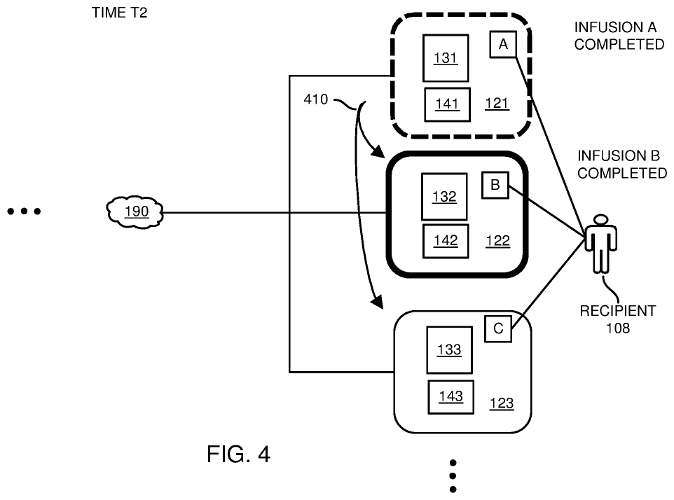

FIG. 4 is an example diagram illustrating communications associated with multiple fluid pumps according to embodiments herein.

At or around time T2, the delivery of fluid A has been completed. In response to completion of delivery, the fluid pump 121 communicates a message 410 to the fluid pump 122. In one embodiment, the message 410 communicated to fluid pump 122 and other fluid pumps 123, etc., indicates the completion of delivering the fluid A to the recipient 108. Note that the message 410 can be generated and received from any suitable resource such as fluid pump 121, order management entity 119, communication device 120, etc.

In accordance with the schedule information 160-1 indicating to start delivery of fluid B after delivery of fluid A has been completed, the message 410 serves as a trigger event in which the fluid pump 122 begins delivery of fluid B to the recipient 108.

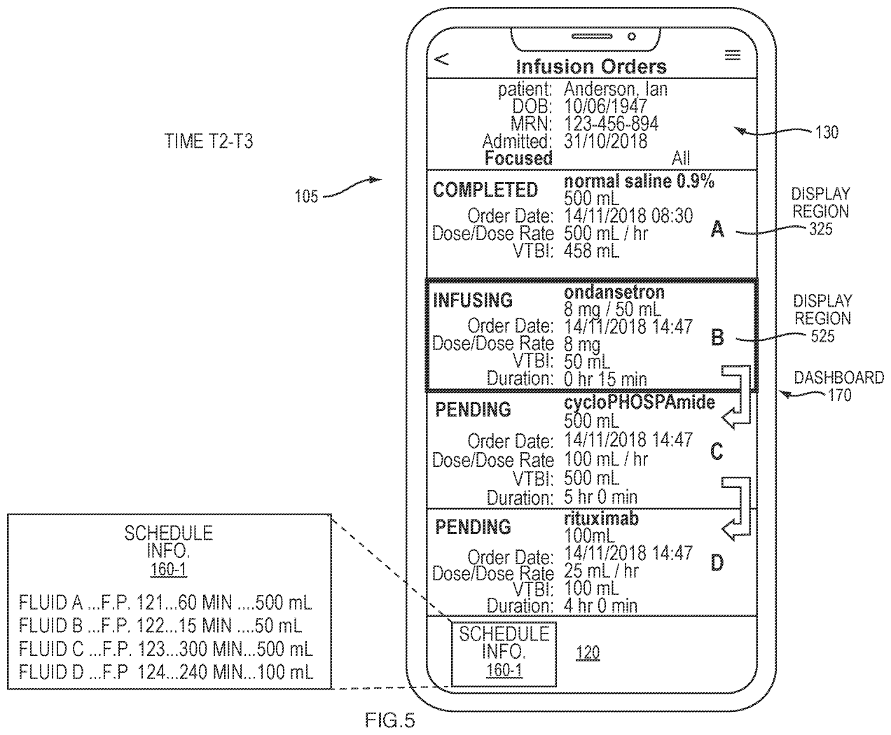

In one embodiment, the fluid pump 122 initiates display of a notice on display screen 132 that the delivery of fluid B has commenced. As further shown in FIG. 5, the communication device displays a notification that the fluid pump 121 completed delivery of the fluid A.

FIG. 5 is an example diagram illustrating display of status information associated with multiple fluid pumps according to embodiments herein.

As previously discussed, at or around time T2, in accordance with the schedule information 160-1 indicating that fluid B infusion is to start on completion of fluid A infusion, the fluid pump automatically (such as without need of further input from other sources) begins delivery of fluid B to the recipient 108 in response to receiving the notification from the fluid pump 121 completing delivery.

In this example embodiment, each of the fluid pumps 121, 122, etc., displays a current status of the pump delivering a respective fluid to the recipient 108. For example, display screen 131 of the fluid pump 121 displays a status (in display region 325) that delivery of fluid A has been completed; display screen 132 of the fluid pump 122 displays a status (in display region 525) of currently delivering fluid B to the recipient 108; and so on.

In further example embodiments, in a manner as previously discussed, each of the fluid pumps (such as via respective controllers) communicate with each other to indicate their status as well as communicate with the order management entity 119 and/or communication device 120 indicating the status of delivering the fluids.

In one embodiment, between time T2 and T3 (time period such as 15 minutes in which to deliver fluid B), the fluid pump 122 repeatedly communicates a status and current progress of the delivery to communication device 120. In response to receiving the input, the communication device 120 displays a notification in display region 525 of the display screen 130 indicating the status of delivering the fluid B. The status information includes any information such as how much or percentage of total fluid that has been delivered at any given time, time remaining until completion of delivering the fluid B, etc.

In one embodiment, the fluid pump 122 also displays a copy of the status information in display region 525 on the display screen 132 of the fluid pump 122.

Thus, the caregiver 107 can view the display screen 130 of the communication device 120 or the respective display screen of each of the fluid pumps to determine a status of delivering the respective fluids.

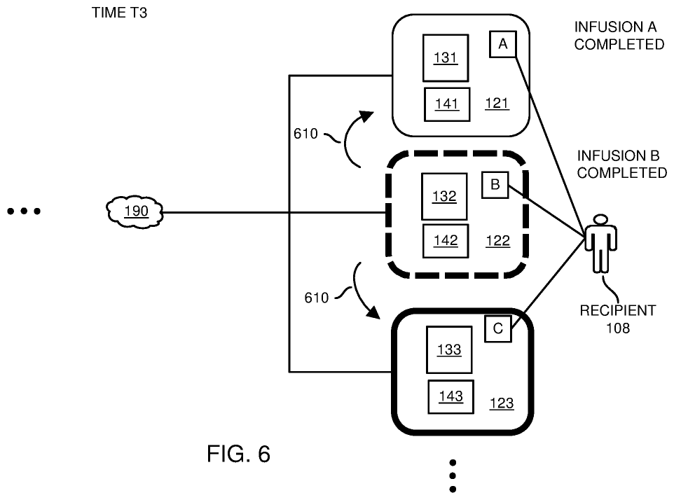

FIG. 6 is an example diagram illustrating communication amongst multiple fluid pumps according to embodiments herein.

At or around time T3, the delivery of fluid B has been completed. In response to completion, the fluid pump 122 communicates a message 610 to the fluid pump 123 and other fluid pumps. In one embodiment, the message 610 communicated to fluid pump 123 and other fluid pumps, indicates the completion of delivering the fluid B to the recipient 108.

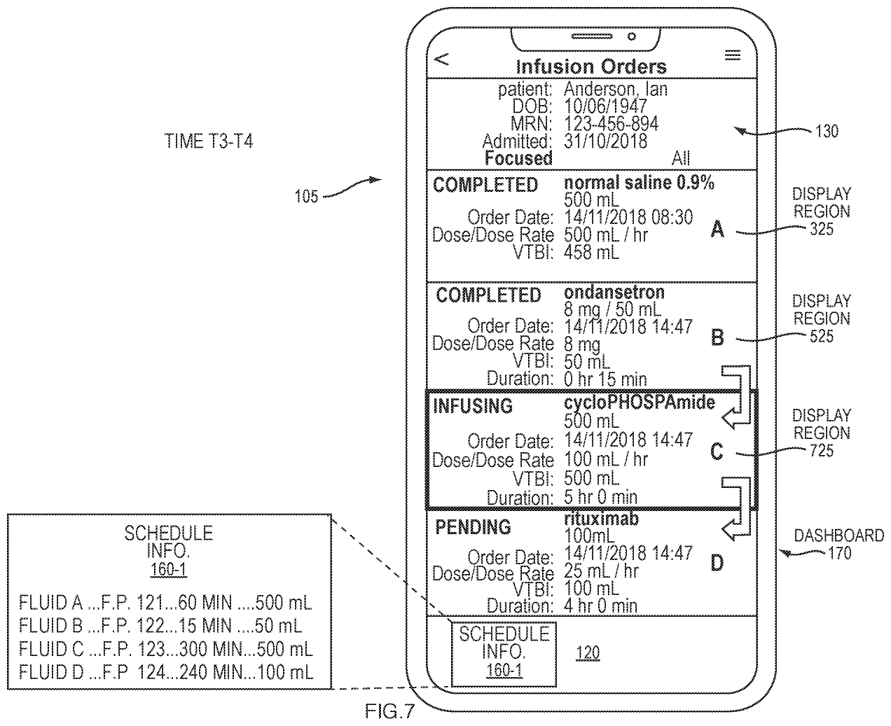

In accordance with the schedule information 160-1, the message 610 serves as a trigger event in which the fluid pump 123 begins delivery of fluid C to the recipient 108. As shown in the following FIG. 7, the fluid pump 122 initiates display of a notice on display screen 133 that the delivery of fluid C has commenced.

FIG. 7 is an example diagram illustrating display of status information associated with multiple fluid pumps according to embodiments herein.

As previously discussed, at or around time T3, in accordance with the schedule information 160-1, the fluid pump 123 automatically begins delivery of fluid C to the recipient 108 in response to receiving the notification from the fluid pump 122 completing delivery of fluid B to the recipient 108.

In this example embodiment, each of the fluid pumps 121, 122, etc., displays a current status of the pump delivering a respective fluid to the recipient 108. For example, display screen 131 of the fluid pump 121 displays a status that delivery of fluid A has been completed; display screen 132 of the fluid pump 122 displays a status that delivery of fluid B has been completed; display screen 133 of the fluid pump 123 displays a status of currently delivering fluid C to the recipient 108; and so on.

In further example embodiments, in a manner as previously discussed, each of the fluid pumps (such as via respective controllers) communicate with each other to indicate their status as well as communicate with the order management entity 119 and/or communication device 120 indicating the status of delivering the fluids.

In one embodiment, between time T3 and T4 (time period such as 300 minutes in which to deliver fluid B), the fluid pump 123 repeatedly communicates a status of the delivery (such as amount delivered over time) to communication device 120. In response to receiving the input, the communication device 120 displays a notification in display region 725 of the display screen 130 indicating the status of delivering the fluid C. In one embodiment, the fluid pump 122 also display information in display region 725 on the display screen 133 of the fluid pump 123.

Thus, the caregiver 107 can view the display screen 130 of the communication device 120 or the display screen of each of the fluid pumps to determine a status of delivering the respective fluids.

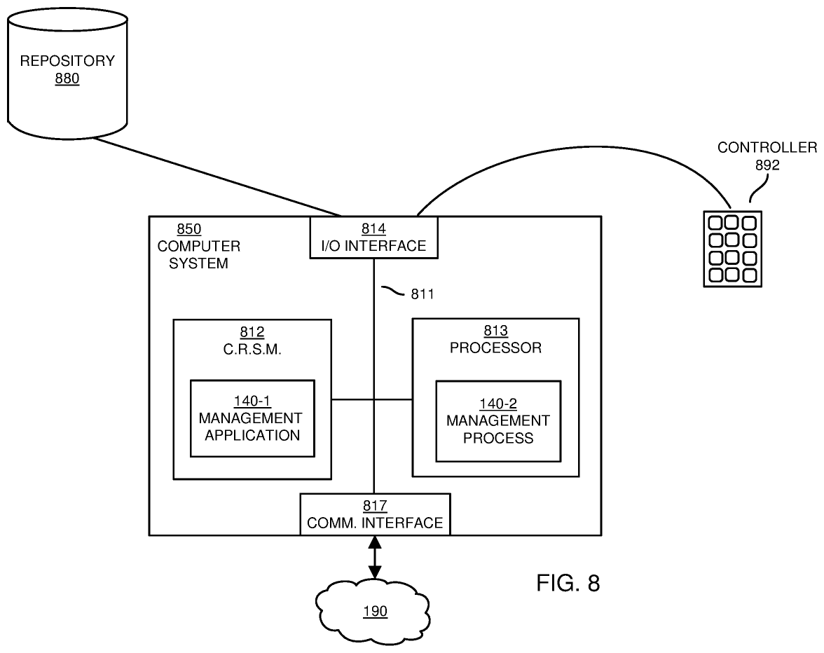

FIG. 8 is an example block diagram of a computer device for implementing any of the operations as discussed herein according to embodiments herein.

In one embodiment, fluid delivery environment 100 includes one or more instances of computer system 850 to execute fluid management system 126, fluid pumps, order management entity 119, controllers 141, 142, and 143, etc.

As shown, computer system 850 of the present example includes an interconnect 811, a processor 813 (such as one or more processor devices, computer processor hardware, etc.), computer readable storage medium 812 (such as hardware storage to store data), I/O interface 814, and communications interface 817.

Interconnect 811 provides connectivity amongst processor 813, computer readable storage media 812, I/O interface 814, and communication interface 817.

I/O interface 814 provides connectivity to a repository 880 and, if present, other devices such as a playback device, display screen, input resource 892, a computer mouse, etc.

Computer readable storage medium 812 (such as a non-transitory hardware medium) can be any hardware storage resource or device such as memory, optical storage, hard drive, rotating disk, etc. In one embodiment, the computer readable storage medium 812 stores instructions executed by processor 813.

Communications interface 817 enables the computer system 850 and processor 813 to communicate over a resource such as network 190 to retrieve information from remote sources and communicate with other computers. I/O interface 814 enables processor 813 to retrieve stored information from repository 180.

As shown, computer readable storage media 812 is encoded with controller application 140-1 (e.g., software, firmware, etc.) executed by processor 813. Controller application 140-1 can be configured to include instructions to implement any of the operations as discussed herein. For example, each of the resources such as resource 119, communication device 120, fluid management system 126, fluid pump 121, fluid pump 122, fluid pump 123, etc., can be configured to include an instance of computer system 850 to execute corresponding operations.

During operation of one embodiment, processor 813 (e.g., computer processor hardware) accesses computer readable storage media 812 via the use of interconnect 811 in order to launch, run, execute, interpret or otherwise perform the instructions in control application 140-1 (i.e., management application) stored on computer readable storage medium 812.

Execution of the management application 140-1 produces processing functionality such as management process 140-2 in processor 813. In other words, the management process 140-2 associated with processor 813 represents one or more aspects of executing management application 140-1 within or upon the processor 813 in the computer system 850.

Those skilled in the art will understand that the computer system 850 can include other processes and/or software and hardware components, such as an operating system that controls allocation and use of hardware resources to execute management application 140-1.

In accordance with different embodiments, note that computer system may be any of various types of devices, including, but not limited to, a wireless access point, a mobile computer, a personal computer system, a wireless device, base station, phone device, desktop computer, laptop, notebook, netbook computer, mainframe computer system, handheld computer, workstation, network computer, application server, storage device, a consumer electronics device such as a camera, camcorder, set top box, mobile device, video game console, handheld video game device, a peripheral device such as a switch, modem, router, or in general any type of computing or electronic device. In one non-limiting example embodiment, the computer system 850 resides in fluid delivery system 100. However, note that computer system 850 may reside at any location or can be included in any suitable resource in network environment 100 to implement functionality as discussed herein.

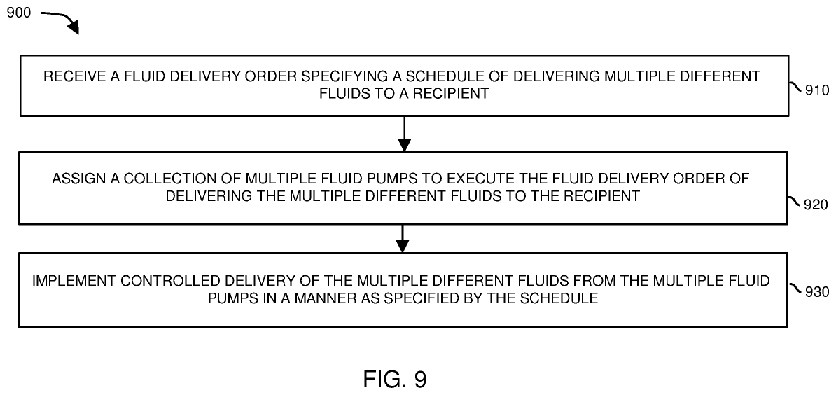

Functionality supported by the different resources will now be discussed via flowcharts in FIG. 9. Note that the steps in the flowcharts below can be executed in any suitable order.

FIG. 9 is a flowchart 900 illustrating an example method according to embodiments. Note that there will be some overlap with respect to concepts as discussed above.

In processing operation 910, the fluid management system 126 and thus caregiver 107 receive a fluid delivery order 105 specifying a fluid therapy of delivering multiple different fluids to a recipient 108.

In processing operation 920, via the fluid management system 126, the caregiver 107 assigns a collection of multiple fluid pumps such as including fluid pump 121, fluid pump 122, fluid pump 123, etc., to execute the fluid delivery order 105 of delivering multiple different fluids A, B, C, etc., to the recipient 108.

In processing operation 930, the fluid management system 126 configures/programs, based on the fluid delivery order 105, and schedule information 160-1, the collection of multiple fluid pumps 121, 122, 123, etc., and corresponding controller 141, 142, 143, etc., to implement controlled delivery of the multiple different fluids A, B, C, etc., from the multiple fluid pumps 121, 122, 123, etc., in a manner as specified by the schedule information 160-1.

Note again that techniques herein are well suited for use in management of fluid delivery systems. However, it should be noted that embodiments herein are not limited to use in such applications and that the techniques discussed herein are well suited for other applications as well.

Based on the description set forth herein, numerous specific details have been set forth to provide a thorough understanding of claimed subject matter. However, it will be understood by those skilled in the art that claimed subject matter may be practiced without these specific details. In other instances, methods, apparatuses, systems, etc., that would be known by one of ordinary skill have not been described in detail so as not to obscure claimed subject matter. Some portions of the detailed description have been presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm as described herein, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has been convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing platform, such as a computer or a similar electronic computing device, that manipulates or transforms data represented as physical electronic or magnetic quantities within memories, registers, or other information storage devices, transmission devices, or display devices of the computing platform.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

We claim:

1. A method comprising:
receiving a fluid delivery order specifying a schedule of delivering multiple different fluids to a recipient;
assigning a collection of multiple fluid pumps to execute the fluid delivery order of delivering the multiple different fluids to the recipient; and
controlling delivery of the multiple different fluids from the multiple fluid pumps in a manner as specified by the schedule.

2. The method as in claim 1, wherein the multiple different fluids include a first fluid and a second fluid;
wherein the multiple fluid pumps assigned to execute the fluid delivery order include a first fluid pump and a second fluid pump; and
wherein the fluid delivery order indicates timing of delivering the second fluid from the second fluid pump with respect to delivering the first fluid from the first fluid pump.

3. The method as in claim 2, wherein the first fluid pump provides notification of completion of delivering the first fluid to the second fluid pump; and
wherein the notification triggers the second fluid pump to deliver the second fluid to the recipient.

4. The method as in claim 1 further comprising:
displaying a visual notification on a first fluid pump of the multiple fluid pumps, the displayed visual notification indicating the schedule of delivering the multiple different fluids from each of the multiple fluid pumps.

5. The method as in claim 1 further comprising:
displaying a notification on a display screen of a first fluid pump of the multiple fluid pumps, the notification indicating triggering conditions in which the second fluid pump is scheduled to deliver the second fluid to the recipient.

6. The method as in claim 1, wherein controlling delivery of the multiple different fluids from the multiple fluid pumps in a manner as specified by the schedule includes:
distributing the schedule to each of the multiple fluid pumps; and
synchronizing operation of the multiple fluid pumps to a common clock.

7. The method as in claim 1 further comprising:
receiving the schedule from a communication device operated by a caregiver, the caregiver producing the schedule, the schedule indicating timing of each of the multiple fluid pumps delivering a respective fluid to the recipient.

8. The method as in claim 1, wherein the multiple different fluids include a first fluid and a second fluid; and
wherein the schedule indicates that delivery of the second fluid to the recipient is contingent upon delivery of the first fluid to the recipient.

9. The method as in claim 8, wherein the schedule indicates to concurrently deliver the first fluid and the second fluid to the recipient.

10. The method as in claim 8, wherein the schedule indicates to sequentially deliver the first fluid and the second fluid to the recipient.

11. The method as in claim 1, wherein the multiple different fluids include a first fluid and a second fluid; and
as specified by the schedule, delivering the second fluid to the recipient after a predetermined delay time following completion of delivery of the first fluid to the recipient.

12. The method as in claim 1, wherein the multiple fluid pumps include a first fluid pump and a second fluid pump; and
wherein assigning the collection of multiple fluid pumps to execute the fluid delivery order includes: via input from a caregiver administering the fluid delivery order: i) selecting the first fluid pump to deliver a first fluid of the multiple different fluids, and ii) selecting the second fluid pump to deliver a second fluid of the multiple different fluids.

13. A system comprising:
multiple fluid pumps including a first fluid pump and a second fluid pump; and
a fluid management system operative to:
receive a fluid delivery order specifying a schedule of delivering multiple different fluids to a recipient;
assign a collection of multiple fluid pumps to execute the fluid delivery order of delivering the multiple different fluids to the recipient; and
control delivery of the multiple different fluids from the multiple fluid pumps in a manner as specified by the schedule.

14. The system as in claim 13, wherein the multiple different fluids include a first fluid and a second fluid;
wherein the multiple fluid pumps assigned to execute the fluid delivery order include a first fluid pump and a second fluid pump; and
wherein the fluid delivery order indicates timing of delivering the second fluid from the second fluid pump with respect to delivering the first fluid from the first fluid pump.

15. The system as in claim 14, wherein the first fluid pump provides notification of completion of delivering the first fluid to the second fluid pump; and
wherein the notification triggers the second fluid pump to deliver the second fluid to the recipient.

16. The system as in claim 13 further comprising:
a display screen operative to display a visual notification on a first fluid pump of the multiple fluid pumps, the displayed visual notification indicating the schedule of delivering the multiple different fluids by each of the multiple fluid pumps.

17. The system as in claim 13 further comprising:
a display screen operative to display a notification indicating triggering conditions in which the second fluid pump is scheduled to deliver the second fluid to the recipient.

18. The system as in claim 13, wherein the controller is further operative to:
control delivery of the multiple different fluids from the multiple fluid pumps in a manner as specified by the schedule, the control including:
distribution of the schedule to each of the multiple fluid pumps; and
synchronizing operation of the multiple fluid pumps to a common clock.

19. The system as in claim 13 wherein the controller is further operative to:
receive the schedule from a communication device operated by a caregiver, the caregiver producing the schedule, the schedule indicating timing of each of the multiple fluid pumps delivering a respective fluid to the recipient.

20. The system as in claim 13, wherein the multiple different fluids include a first fluid and a second fluid; and
wherein the schedule indicates that delivery of the second fluid to the recipient is contingent upon delivery of the first fluid to the recipient.

21. The system as in claim 20, wherein the schedule indicates to concurrently deliver the first fluid and the second fluid to the recipient.

22. The system as in claim 20, wherein the schedule indicates to sequentially deliver the first fluid and the second fluid to the recipient.

23. The system as in claim 13, wherein the multiple different fluids include a first fluid and a second fluid; and
as specified by the schedule, delivering the second fluid to the recipient after a predetermined delay time following completion of delivery of the first fluid to the recipient.

24. The system as in claim 13, wherein the controller is further operative to:
via input from a caregiver administering the fluid delivery order: i) select the first fluid pump to deliver a first fluid of the multiple different fluids, and ii) select the second fluid pump to deliver a second fluid of the multiple different fluids.

25. Computer-readable storage hardware having instructions stored thereon, the instructions, when carried out by computer processor hardware, causes the computer processor hardware to:
receive a fluid delivery order specifying a schedule of delivering multiple different fluids to a recipient;
assign a collection of multiple fluid pumps to execute the fluid delivery order of delivering the multiple different fluids to the recipient; and
control delivery of the multiple different fluids from the multiple fluid pumps in a manner as specified by the schedule.
